(19) United States
(12) Patent Application Publication
(10) Pub. No.: US 2009/0017562 A1
(43) Pub. Date: Jan. 15, 2009

(54) RAMAN-ACTIVE REAGENTS

(75) Inventors: Marc D. Porter, Salt Lake City, UT (US); Hye-Young Park, Yongin Si Suji-gu (KR); Robert J. Lipert, Ames, IA (US)

Correspondence Address: MCKEE, VOORHEES & SEASE, P.L.C., 801 GRAND AVENUE, SUITE 3200, DES MOINES, IA, 50309-2721, US

(73) Assignee: IOWA STATE UNIVERSITY RESEARCH FOUNDATION, INC., Ames, IA (US)

(21) Appl. No.: 12/044,551

(22) Filed: Mar. 7, 2008

Related U.S. Application Data
(60) Provisional application No. 60/894,569, filed on Mar. 13, 2007.

(51) Int. Cl. G01N 33/544; G01N 33/543

(52) U.S. Cl. 436/528; 436/518

(57) ABSTRACT

Raman Active Reagents (ERLs) are developed which use a nanoparticle substrate substantially covered with a mixed monolayer derived from a Raman active reporter molecule and an analyte binding molecule that both bind to the surface of the nanoparticle and thereby avoid the necessity for separate synthesis of a bifunctional linker molecule in making the ERL.

### CROSS REFERENCE TO RELATED APPLICATIONS

This application claims priority under 35 U.S.C. § 120 to provisional application Ser. No. 60/894,569 filed Mar. 13, 2007, herein incorporated by reference in its entirety.

### GRANT REFERENCE

This invention was made with government support under Contract No. MDA972-02-2-0002 awarded by CEROS under DARPA. The government may have certain rights in the invention.

### BACKGROUND OF THE INVENTION

Many assays exist for detecting and measuring analytes of small quantity in the presence of a large volume of other substances. Such assays typically make use of the high binding affinity between the analyte (the substance to be detected or measured) and a second molecule having a high degree of specificity for binding to that analyte. These assays are often referred to as ligand-binding assays.

One of the most common ligand-binding assays are immunoassays. Immunoassays typically employ an antigen and an antibody which specifically binds to the antigen to form an antibody/antigen complex. In order to measure the extent of the antibody/antigen binding, one member of the complex is generally labeled or tagged with a traceable substance. The presence of the traceable substance, and hence the presence of the antibody or antigen to which it is attached, may then be detected or measured using a variety of different techniques depending upon the unique characteristics of the label employed. These techniques may include an analysis and measurement of scintillation counting, fluorescence, absorption, electrochemistry, chemiluminescence, Rayleigh scattering and Raman scattering. Of these techniques, fluorescence spectroscopy has been one of the most widely used readout methods, primarily because of its high sensitivity.

Although fluorescence spectroscopy has seen substantial use in scientific research and clinical diagnostics, there are disadvantages in using fluorescence spectroscopy. For instance, the different types of fluorescent molecules used in fluorescence spectroscopy typically require excitation with photons of differing wavelengths. Therefore, if the detection of multiple fluorescent molecules is desired in a single sample, multiple light sources may be required. If a single light source is used, there will often exist a spectral overlap between the emission of the different fluorescent molecules such that reliable individual and quantitative detection of multiple analytes in a single sample is limited.

Today, many assays require the concomitant determination of more than one analyte in a single test sample (e.g., the screening of cancer markers, such as a-fetoprotein and carcinoembryonic antigen). There are two general approaches to assaying multiple analytes in a single sample. One approach immobilizes different binding molecules on a solid support at spatially separated addresses. Multiple analytes can then be detected using the same label, with identification based on address location. Alternatively, different labels can be used to detect multiple analytes simultaneously in the same spatial area. In this case, each analyte obtains its own distinct label.

These inventors have explored Raman spectroscopy as an alternative to fluorescence spectroscopy. Raman spectroscopy measures the level of Raman scattering induced by the application of a radiation source, i.e. light source, on an analyte. The light incident on the analyte is scattered due to excitation of electrons in the analyte. “Raman” scattering occurs when the excited electron returns to an energy level other than that from which it came, resulting in a change in the wavelength of the scattered light and giving rise to a series of spectral lines at both higher and lower frequencies than that of the incident light. The series of spectral lines is generally called, “the Raman spectrum”.

Conventional Raman spectroscopy usually lacks sufficient sensitivity for use as a readout method for immunoassays. Raman spectroscopy is also unsuccessful for fluorescent materials due to the fact that broad fluorescence emission bands tend to swamp the weaker Raman bands.

However, a modified form of Raman spectroscopy based on “surface enhanced” Raman scattering (SERS) has proved to be more sensitive and thus of more general use. In the SERS form of Raman spectroscopy, the analyte whose spectrum is being recorded is closely associated with a roughened metal surface. This close association leads to a large increase in detection sensitivity, the effect being greater the closer the analyte sits to the metal surface.

The manner in which surface enhancement occurs is not yet fully understood, but it is thought that the incident light excites conduction electrons in roughened metal surfaces or particles, generating a plasma resonance (plasmon). As a result, the electromagnetic field in the vicinity of the metal surface is greatly amplified, giving rise to enhanced Raman scattering in molecules located close to the surface.

Surprisingly, there have been only a few reports on the application of SERS for detection in immunoassays. Two of these approaches used a sandwich-type assay, with coupled surface and resonance enhancements. In particular, Rohr et al., Anal. Biochem. 1989, 182, 388, used labeled detection antibodies and roughened silver films coated with a capture antibody (see also U.S. Pat. No. 5,266,498 to Tarch et al.), and Dou et al., Anal. Chem. 1997, 69, 1492, exploited the adsorption on silver colloids of an enzymatically amplified product. Another approach by White et al., International Application Publication No. WO 99/44065, employs an immunoassay based on the displacement of SERS and surface enhanced resonance Raman (SERRS) active analyte analogs which are modified so as to have particular SERS and SERRS surface seeking properties. Upon introduction of a sample, the analyte analogs are displaced by the analyte of interest in the sample and exposed to a SERS or SERRS surface, such as an etched or roughened surface, a metal sol or an aggregation of metal colloid particles. Raman spectroscopy is then performed to detect the displaced analyte analog associated with the SERS or SERRS surface to determine the presence or quantity of the analyte in the sample.

A major barrier that prohibits using SERS for the direct detection of biological samples is that the surface enhancement effect diminishes rapidly with increasing distance from the metallic surfaces. In other words, strong SERS signals are observed only if the scattering centers are brought into close proximity (<100 nm) to the surface. In addition, although Raman spectra of biomolecules can be obtained on silver surfaces when coupling SERS and resonance enhanced scattering, the spectra are usually lacking of sufficient chemical content and/or signal amplitude to be used for immunoassay purposes.

In previous applications filed by some of these same inventors, Ser. No. 09/961,628 filed Sep. 24, 2001 and Ser. No. 10/931,142 filed Aug. 31, 2004, (herein incorporated by reference), some of these barriers we overcome by developing a novel class of Raman-active reagents having both Raman-active reporter molecules and binding molecules integrated with each other on the same SERS surface. In the prior art, the SERS or SERRS surface and the Raman-active molecule are not normally integrated with each other, but are merely placed in close proximity to each other by the combination of an analyte sandwiched between an antibody immobilized on the enhancing surface and an antibody attached to a Raman active molecule, or as in our earlier applications, the combination of the SERS or SERRS surface with a particular SERS or SERRS surface seeking group coupled to an analyte analog and a Raman-active molecule, after exposure to the sample.

Those earlier applications presented as a novel class of Raman-active reagents for use in biological and other applications, as well as methods and kits for their use and manufacture. They relied upon bifunctional linker molecules that also functioned as Raman-active labels.

The Raman-active reagents each included a Raman-active reporter molecule, a binding molecule, and a surface enhancing particle capable of causing surface enhanced Raman scattering. The Raman-active reporter molecule and the binding molecule were operably linked to the particle to give both a strong surface enhanced Raman scattering (SERS) signal, and to provide biological functionality, i.e. antigen or drug recognition. The Raman-active reporter molecule and the binding molecule were either directly linked to the surface enhancing particle or indirectly linked to the surface enhancing particle by way of a linker molecule. In one embodiment, the Raman-active reporter molecule and the binding molecule were each independently linked to the surface enhancing particle. In a second embodiment, the binding molecule was operably linked to the Raman-active reporter molecule, which is also operably linked to the surface enhancing particle.

The Raman-active reagents of this present invention serve as an alternative to the reagents earlier described, which minimizes the need for bifunctional molecule preparation.

Self-assembled monolayers (SAMs) have current widespread use in the detection art. There are many SAM systems, such as organoalkanethiolate on gold or silver, organosilicon on oxides, and carboxylic acid on metal oxides. Among them, SAMs on gold is the most studied experimentally and theoretically. Alkanethiolates are generally composed of three regions: a sulfur head group, a polymethylene or aromatic spacer group, and an end or terminal group (FIG. 1). Thiols chemisorb to gold via the sulfur head group while the alkyl chain provides additional stability from interchain van der Waals or π-π stacking forces, leading to well-ordered 2D structures. The surface characteristics of SAMs are typically controlled by the end group functionality, which can be readily varied synthetically. Because of the ability to modify its surface in one simple step, SAMs on gold have been widely used as a model of bio-surfaces as well as platform for sensor construction.

Mixed SAMs serve as an experimental system to study interactions of biomolecules with surfaces by tailoring the surface chemical and structural properties. They can also provide means to control gradients of composition, which can also be of value in studies of biomolecules adsorption and manipulation. Mixed SAMs can be formed by co-adsorption from thiol or disulfide mixtures, or by adsorption of asymmetric disulfides. Studies show that the homogeneity and preferential adsorption of these precursors can be affected by chain length, head group, tail group, and solvent. In studies when two components with different chain lengths were used, the mixed monolayer phase segregated due to a thermodynamically controlled process. In ethanol, the favorable adsorption of one component over the other was controlled by solubility and ability to form intra-monolayer hydrogen bonds.

Since we have demonstrated Extrinsic Raman Labels (ERLs) have strong potential as an analytical tool, as evidenced by our previous applications, we now have demonstrated a system using ERL's and SAMs. This strategy exploits the strong surface enhanced Raman scattering (SERS)-derived signal from organic dyes (i.e., reporter molecules) that are immobilized on Au nanoparticles along with the appropriate chemical and biospecific species. The identify of each antigen is determined from the characteristic SERS spectrum of the nanoparticle-bound reporter species linked to the tracer antibody, with each antigen then quantified by the spectral intensity of reporter species. The advantages of this strategy which uses self-assembled monolayers created by covering substrate (gold) nanoparticles with two compounds, one which binds the analyte to the particle, and one which binds a reporter to the molecule, is that special synthesis steps and their attendant expense are avoided. This then is the primary object of the invention.

### BRIEF SUMMARY OF THE INVENTION

Raman Active Reagents (ERLs) are developed which use a nanoparticle substrate substantially covered with a mixed monolayer derived from a Raman active reporter molecule and an analyte binding molecule that both bind to the surface of the nanoparticle and thereby avoid the necessity for separate synthesis of a bifunctional linker molecule in making the ERL.

### BRIEF DESCRIPTION OF THE DRAWINGS

FIG. 1 is an idealized representation of self-assembled alkanethiolate monolayer on gold.

FIG. 2 represents various ERL constructs, with FIG. 2A showing co-mobilization, FIG. 2B showing separate use of bifunctional compounds and FIG. 2C showing mixed monolayer ERL constructs of the invention.

FIG. 3 presents representative SERS spectra for assays of (A) human IgG, (B) mouse IgG, and (C) E. herbicola.

FIG. 4 similarly shows SERS intensity for the spectra of FIG. 3.

FIG. 5 shows SERS spectra of (A) a single labeled E. coli 0157:H7 cell, and (B) a blank area.

### DETAILED DESCRIPTION OF THE PREFERRED EMBODIMENT

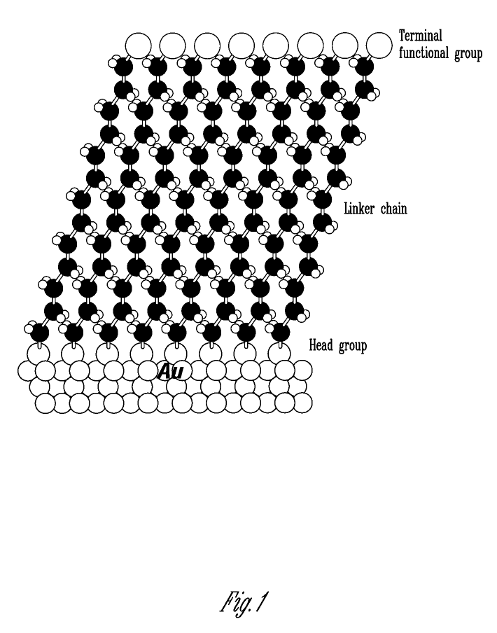

FIG. 1 is an idealized representation of self assembled alkanethiolate monolayer on gold, presented to illustrate why SAMs on gold is a widely used model of bio-surfaces for sensor construction.

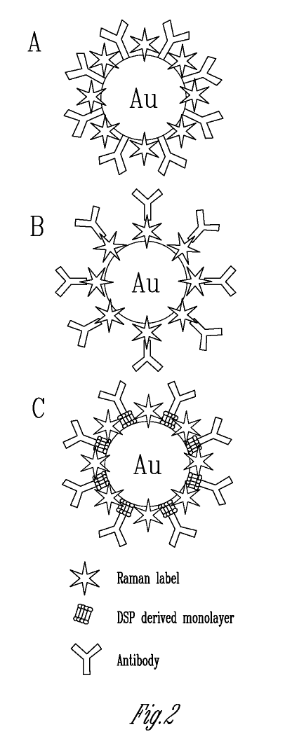

FIG. 2A depicts an example of an earlier type of ERL™. While successfully applied to the concurrent qualitative analysis of two biolytes (i.e., rat and rabbit IgG), questions remained regarding a contribution to the apparent nonspecific adsorption of the ERLs™ by the possible transfer of weakly adsorbed antibodies from one ERL™ to another ERL™ that had been modified with a distinctly different antibody coating. There could therefore be the possibility of “cross-talk” between different ERLs present in the same solution during a multiplexed labeling step. This approach was also complicated by occasional problems with the stability of the particle suspension, also potentially the result of the desorption of the protein-based coating.

The next scheme developed in our process towards the present invention is shown in FIG. 2B. It is described in the earlier incorporated by reference applications and used a bifunctional Raman reporter molecule to covalently couple the antibody to the particles. This scheme improved particle stability and reduced the limit of detection via a lower level of non-specific ERL™ adsorption. Using this type of ERL™, we recently reported on the femtomolar detection of prostate specific antigen (PSA) directly in human serum. This approach, while working with a high level of effectiveness, nevertheless required the separate synthesis of the bifunctional reporters. Bifunctional reports as illustrated in FIG. 2B therefore work, but are expensive and are not normally commercially available which requires synthesis along the way to running the detection analysis. This adds time and expense. It therefore demonstrates the unfilled need to develop a system which eliminates the need for a separate step requiring synthesis of bifunctional reporter molecules.

FIG. 2C introduces a new design for ERLs™ that does eliminate the need for separate synthesis of a bifunctional reporter, and yet gets sensitive detection results. In this scheme, the surface of gold nanoparticles is modified, for example, with two different thiolates, each derived from commercially available compounds. One thiolate component has a large Raman cross section and serves as the reporter molecule. The other component is derived from the bifunctional compound dithiobis (succinimidyl propionate) (DSP), which has both a disulfide and a succinimidyl functional group for the respective chemisorption onto gold and the facile covalent coupling of antibodies to the particle. DSP, however, is an intrinsically weak Raman scatterer. This scheme therefore facilitates the production of distinctive ERLs™, referred to hereafter as mixed-monolayer ERLs, for the potential use in multianalyte assays.

### EXAMPLES

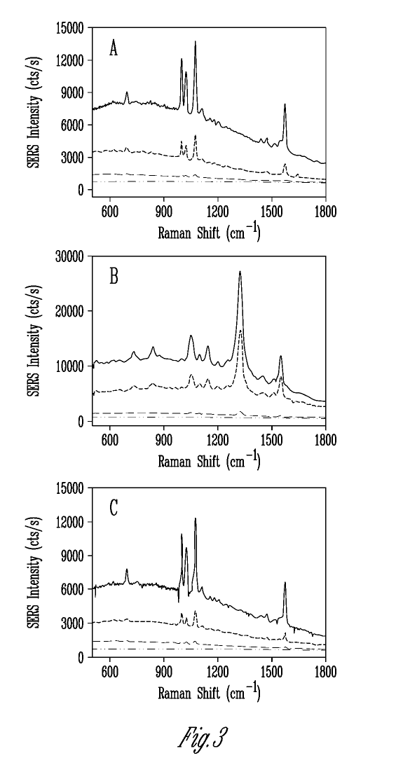

To test the effectiveness of this concept, mixed monolayer ERLs™ were constructed using thiophenol (TP), mercaptobenzoic acid (MBA), and dithiobis succinimidyl nitrobenzoate (DNBA) as Raman reporters and DSP as the coupler. FIG. 3 presents representative SERS spectra for assays of (A) human IgG, (B) mouse IgG, and (C) E. herbicola. Each set of data was obtained using the appropriate capture substrate, prepared by the procedures described earlier. As expected, the spectra in FIGS. 2A, C exhibit distinctive peaks for TP, with the respective signal strengths increasing as the concentration of human IgG and E. herbicola increases. All the observed bands (999, 1022, 1069, and 1568 cm⁻¹) are from aromatic ring modes of the TP label. This demonstrates the facility with which the biospecificity of the ERL's can be changed without spectral interference. The assay of mouse IgG used 5,5′-dithiobis (2-nitrobenzaote) (DNBA), in contrast, as the reporter. These results are given in FIG. 2B. These spectra also undergo an increase in signal strength with antigen concentration. Note how a distinctive spectral signal is obtained by simply changing the Ramon-active component of the mixed monolayer. We add that the spectrum for the DNBA-based assay is virtually identical to that for the DSNB-derived spectrum, which reflects the use of DNBA as the starting material in the synthesis of DSNB. This demonstrates the effectiveness of the mixed monolayer, wherein the biospecificity and spectral identity can be changed to generate a wide variety of bioanalytical reagents.

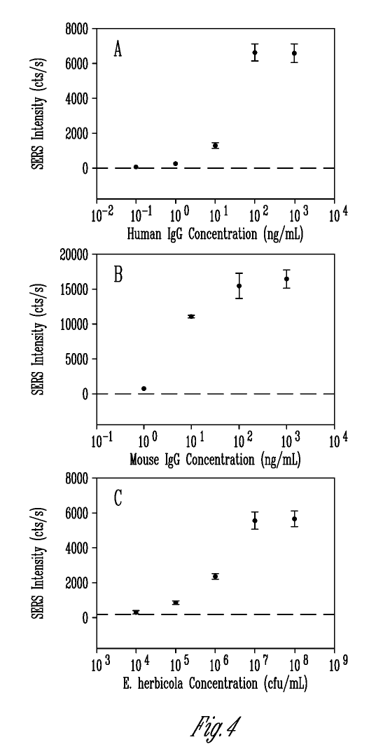

These spectra were used to construct the dose response curves shown in FIG. 4. The plots for the assays of human IgG and E. herbicola employed the peak at 1069 cm⁻¹, whereas that for mouse IgG utilized the peak at 1336 cm⁻¹1. Each data point represents the average of five different measurements. Spot-to-spot variation was ˜10%. Limits of detections were estimated at 0.06 ng/mL for human IgG, 0.04 ng/mL for mouse IgG, and 10⁴ cfu/mL for E. herbicola. The LOD for E. herbicola is about the same as was measured using the bifunctional reporter DSNB. This clearly shows that the mixed monolayer ERL™ approach is successfully applied to detection of bacteria and proteins without losing performance. With excellent particle stability and relatively simple preparation, the mixed monolayer ERL™ shows potential to be used not only for single analytes but also for multi analyte assays for various types of biomolecules.

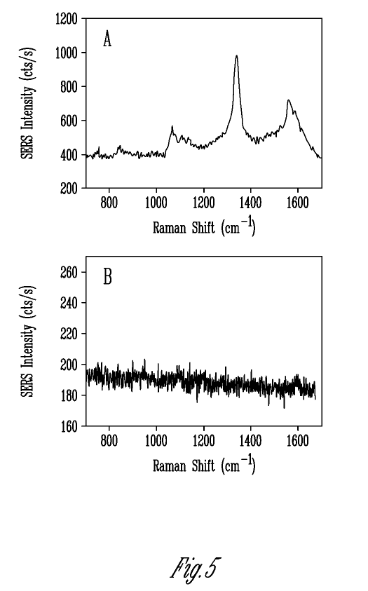

Single E. coli O157:H7 SERS. The SERS signal from a single E. coli O157:H7 cell was measured using a SERS microscope. After completing the sandwich immunoassay utilizing DSNB-based ERLs™, the laser beam, focused to a spot 2.5-3 μm in diameter, was placed onto a single E. coli O157:H7 cell tagged with ERLs™. Since the size of the laser spot size is comparable to that of E. coli O157:H7, the observed signal originates primarily from the irradiated cell and not other portions of the capture substrate. A strikingly large signal from a single bacterium is evident. (FIG. 5A) On the other hand, no signal was observed on the area (FIG. 5B) without E. coli O157:H7, further demonstrating the selectivity of our ERLs™. In an earlier single particle SERS study, 80 nm DSNB-coated particles gave a SERS signal of ˜6 counts/s/particle using the same instrument setup. The signal of ˜600 counts/s from a single cell, therefore, suggests that the cell is covered with many particles. Moreover, given the large size of E. coli O157:H7 cells, it is not expected that ERLs™ captured on the top surface of the cells will contribute strongly to the SERS signal, based on the importance of particle-substrate electromagnetic coupling in producing the enhanced Raman scattering in these experiments and the rapid decay of this coupling as the particle-substrate separation distance increases. We estimate that in the previous study, ERLs™ were located somewhere between 10 to 20 nm from the metal substrate.

The mixed-monolayer ERLs can be prepared in relatively simple steps using commercially available materials, i.e., synthesis of bifunctional reporter is not required, facilitating the generation of ERLs with different Raman reporter molecules for multiplexed applications. It is shown that ERLs based on mixed monolayers have a comparable level of performance compared to ERLs fabricated with bifunctional reporter molecules.

The reagents for the mixed monolayer can be from a variety of compounds. For binding the antibody, any disulfide terminating in a succinimide group will work, e.g., DSP, DSU. It is preferable that the molecule not have a strong Raman scattering center, so aromatic moieties should be avoided. Suitable examples include N-hydroxysuccinimide esters, N-hydroxysulfosuccinimide esters, maleimide, and hydrazide. For the reporter molecule, thiols are preferred. They are usually benzyl or naphthyl based, and can be obtained commercially at high purity with low cost. Others that work include disulfide, isocyanide, phosphino, carboxylate, or diazonium salt. Again, thiol and disulfide are the preferred compounds.

For the reporter component of the mixture, in addition to the above, the compounds should contain substituted aromatic moieties. For example, substituted benzene or naphthalene groups, with the substituents being chosen from the following: hydrogen, halide, nitro, nitrile, carboxylate, aldehyde, ester, ether, or alcohol groups.

All of the above reagents may be supplied in kits to perform assays with appropriate instructions to prepare the mixed monolayer ERL.

What is claimed is:

1. A Raman active reagent comprising;
a surface enhanced particle capable of causing surface enhanced Raman scattering;
said surface enhanced particle being substantially covered with a mixed monolayer derived from a Raman active reporter molecule and a binding molecule capable of binding to both the surface enhanced particle, and an antibody;
said reporter molecule being capable of providing a measurable Raman scattering signal when illuminated by an excitation source capable of inducing Raman scattering.

2. The reagent of claim 1 wherein the mixed monolayer is derived from two different thiols, one having bifunctionality.

3. The reagent of claim 2 wherein the one thiol having bifunctionality has a disulfide functional group and a succinimidyl functional group.

4. The reagent of claim 3 wherein the bifunctional thiol is dithiobis (succinimidyl propionate) (DSP).

5. The reagent of claim 1 supplied in a kit with instructions to prepare the reagent.

6. A method of preparing a Raman active reagent, comprising:
covering a nanoparticle of a material capable of causing surface enhanced Raman scattering with a mixed monolayer derived from a Raman active reporter molecule and an analyte binding molecule.

7. The method of claim 6 wherein the analyte binding molecule is an organic non-aromatic disulfide terminating in a succinimide moiety.

8. The method of claim 6 wherein the reporter molecule is a thiol selected from the group consisting of benzyl or naphyl based thiols.
